(19) United States
(12) Patent Application Publication
(10) Pub. No.: US 2004/0156844 A1
(43) Pub. Date: Aug. 12, 2004

(54) Tamper resistant oral dosage form

(76) Inventor: Curtis Wright, Norwalk, CT (US)

Correspondence Address: DARBY & DARBY P.C., P. O. BOX 5257, NEW YORK, NY, 10150-5257, US

(21) Appl. No.: 10/478,879

(22) PCT Filed: May 22, 2002
(86) PCT No.: PCT/US02/16269

(51) Int. Cl. A61K 39/395

(52) U.S. Cl. 424/132.1

(57) ABSTRACT

The present invention provides an oral dosage form including a medicament and an insoluble component adhered to or encapsulates the medicament that is susceptible to cleavage by colonic bacteria and resistant to cleavage by intestinal saccharidases.

### CROSS REFERENCE TO RELATED APPLICATIONS

Reference is made to U.S. application serial No. 60/292,603, filed May 22, 2001, which is incorporated herein by reference.

### FIELD OF THE INVENTION

The present invention relates to a dosage form including substances adhered to the therapeutic agent which are soluble in the gastrointestinal tract. In particular, the dosage forms of the invention are either encapsulated with or modified by acid labile substances or linkages that are particularly susceptible to gastric enzymes. Thus the therapeutic agent in the dosage form is unavailable unless taken as directed.

### BACKGROUND OF THE INVENTION

Approximately 90% of all drugs used to produce a systemic effect are administered via the oral route. Of those administered orally, tablets are preferred for a variety of reasons. For example, tablets are unit dose forms that offer the greatest uniformity of content, in a light and compact package. In addition, tablets are relatively simple and inexpensive to produce, package, and ship. Tablets also offer the greatest ease of swallowing, especially when coated, they easily lend themselves to certain special-release profile products, such as enteric or delayed-release products, and they have the best combined properties of chemical, mechanical, and microbiological stability of all oral dosage forms. For at least these reasons, a wide variety of medications are currently available in tablet form. However, oral dosage forms are not free from abuse, especially certain analgesics that are capable of rapid pain relief with a simultaneous euphoric effect.

Enteric coatings have been used to modify drug release in oral dosage forms. Enteric coatings are designed to remain intact in the stomach, but will dissolve and release the contents of the dosage form in the small intestine. The coatings are generally used to delay the release of drugs that are inactivated by the stomach contents or that may irritate the gastric mucosa, thereby causing nausea or bleeding. The action of enteric coatings results from a difference in composition of the gastric and intestinal environments with respect to pH and the presence of endogenous enzymes. Most enteric coatings are formulated to remain intact in the low pH of the stomach, but readily dissolve when the pH rises to about 4-5. The most effective enteric polymers are poly acids having a pKa of 3-5. See Remington: The Science and Practice of Pharmacy 9th Ed., Ed. Alfonso R. Gennaro, et al., Philadelphia College of Pharmacy and Science (1995), p. 1653 et seq. However, enteric coatings have not been used to deter abuse and provide a tamper resistant dosage form.

U.S. Pat. No. 5,840,332 to Lerner et al. provides a gastrointestinal delivery system that includes a drug in combination with a core material that is surrounded by a water insoluble coating. When the delivery device enters the gastrointestinal tract, the dosage form absorbs liquid, thereby forming channels interconnecting the drug-containing core with the outside of the delivery device. These channels therefore, allow the release of the drug from the core into the gastrointestinal tract.

U.S. Pat. No. 5,849,327 to Berliner et al. describes a dosage form that permits delivery of a therapeutic agent to the lower gastrointestinal tract. The dosage form is oral ingested and contains a plurality of porous microscopic beads, wherein the therapeutic agent is contained within the pores and the pores are plugged with a polysaccharide that is chemically degradable by colonic-specific bacteria. The dosage form also contains an enteric coating that insures that the dosage form remains intact until it reaches the lower gastrointestinal system.

However, the prior art dosage forms do not address the unique problems posed by therapeutic agents prone to abuse, such that when taken as directed, the therapeutic agent is delivered properly, but when abused, the normal delivery mechanism is circumvented or interrupted to prevent abuse by an unintended user. The present invention addresses these problems by encapsulating or covalently modifying the therapeutic agent with an insoluble component that is cleaved by enzymes endogenous to the gastrointestinal tract, but which is not susceptible to cleavage by intestinal saccharidases.

### SUMMARY OF THE INVENTION

The present invention overcomes the problems posed by the abuse of oral dosage forms by providing the therapeutic agent in a form that renders the therapeutic agent insoluble outside the gastrointestinal tract. Therefore, the oral dosage form of the present invention does not release the therapeutic agent unless and until the dosage form is properly ingested.

Accordingly, the present invention provides an oral dosage form consisting of a therapeutic agent and an insoluble component adhered to the therapeutic agent, i.e., encapsulating or linked to the therapeutic agent, such that the dosage form is insoluble outside of the gastrointestinal tract. In one embodiment, the insoluble component is covalently linked to the therapeutic agent, and optionally, the covalent linkage is a chemical bond that is susceptible to cleavage by colonic bacteria, but not susceptible to cleavage by intestinal saccharidases.

In an alternative embodiment, the present invention provides a dosage form in which an insoluble component encapsulates the therapeutic agent. For example, the insoluble component may be a protein that is susceptible to proteolytic digestion by enzymes endogenous to the gastrointestinal tract. In a preferred embodiment, the protein is an immunoglobulin construct, e.g., a synthetic antibody. Alternatively, the therapeutic agent is dispersed in a pellet, bead, or microparticle, and the pellet, bead or microparticle is encapsulated by the protein. When such a dosage form is used improperly, e.g., crushed for inhalation, transmucosal or parenteral abuse, an insoluble gel is formed that prevents absorption of the therapeutic agent.

Also provided are methods of administering therapeutic agents and methods of formulating therapeutic agents that are insoluble outside of the gastrointestinal tract.

### DESCRIPTION OF THE INVENTION

The dosage form of the present invention includes a high surface area suspension of particles, film, gel or other physical substrate that is insoluble and encapsulates or is linked to the active therapeutic agent and renders it insoluble outside of the gastrointestinal tract. Thus, in vitro solubilization of the dosage form is difficult and/or time consuming because the dosage form must interact with the patient's gastrointestinal tract to release the therapeutic agent. Thus, the delayed or controlled release profile of the dosage form prevents or limits the potential for abuse by improper use.

The dosage forms of the present invention are soluble in the gastrointestinal tract, i.e., in the stomach and intestine, and generally insoluble elsewhere due to encapsulation of or attachment to a therapeutic agent a substance which is acid labile or which contains an acid labile linkage. Once exposed to the acidic environment of the gastrointestinal tract (pH less than 4), the substance is released from the therapeutic, i.e., either the acid labile linkage is cleared or the acid labile substance is degraded and the therapeutic agent is revealed.

The gastrointestinal tract contains certain enteric peptidases that are not generally present elsewhere in the human body because they are optimally active only in an acidic environment and the dosage forms of the invention include substances (encapsulating or linked to the therapeutic) that are susceptible to enteric peptidases but which are not degraded by intestinal saccharides. For example, acid proteases, such as pepsin, are present in gastric juice and are optimally active at a pH between 2 and 3. Pepsin contains two active site aspartate residues and one of which must be ionized for the enzyme to be active. Two other digestive enzymes are trypsin and elastase. Thus, in one embodiment, the substances used to coat the therapeutic of the invention are degraded by or contain linkages that are cleared by pepsin, trypsin, or elastase.

Therefore, in a first embodiment of the present invention the dosage form consists of a therapeutic agent and an insoluble component adhered to the therapeutic agent, such that the dosage form is insoluble outside of the gastrointestinal tract. In a preferred embodiment, the insoluble component is covalently linked to the therapeutic agent, and more preferably, the covalent linkage is a chemical bond that is susceptible to cleavage by colonic bacteria, but not susceptible to cleavage by intestinal saccharidases. In a preferred embodiment, the linkage is a an acid labile ester or amide linkage, a carbohydrate linkage, or a sulfonate or glucuronide.

In an alternative embodiment, the dosage form contains an insoluble component that encapsulates the therapeutic agent which is acid labile. For example, the insoluble component may be a protein that is susceptible to proteolytic digestion by enzymes endogenous to the gastrointestinal tract. In a preferred embodiment, the protein is an immunoglobulin construct, e.g., a synthetic antibody. Alternatively, the therapeutic agent is dispersed in a pellet, bead, or microparticle, and the pellet, bead or microparticle is acid labile.

While the dosage form may be formulated for virtually any medicament, in a preferred embodiment, the therapeutic agent are fentanyl, buprenorphine, etorphine and related opioids, or any combinations thereof.

The present invention may be formulated into a pharmaceutical composition. The pharmaceutical composition also may include additives, such as a pharmaceutically acceptable carrier, a flavorant, a sweetener, a preservative, a dye, a binder, a suspending agent, a dispersing agent, a colorant, a disintegrant, an excipient, a diluent, a lubricant, a plasticizer, an edible oil or any combination of any of the foregoing.

Suitable pharmaceutically acceptable carriers include, but are not limited to, ethanol; water; glycerol; aloe vera gel; allantoin; glycerin; vitamin A and E oils; mineral oil; PPG2 myristyl propionate; vegetable oils and solketal. Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; polyethylene glycol; waxes; and the like. Suitable disintegrators include, but are not limited to, starch such as corn starch, methyl cellulose, agar, bentonite, xanthan gum and the like. Suitable lubricants include, but are not limited to, sodium oleate, sodium stearate, magnesium stearate, sodium benzoate, sodium acetate, sodium chloride and the like. A suitable suspending agent is, but is not limited to, bentoite. Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone and gelatin. Suitable edible oils include, but are not limited to, cottonseed oil, sesame oil, coconut oil and peanut oil. A suitable pharmaceutical diluent is, but is not limited to, water. Examples of additional additives include, but are not limited to, sorbitol; talc; stearic acid; and dicalcium phosphate.

The pharmaceutical compositions may be formulated as solid unit dosage forms, such as tablets, pills, and capsules. Unit dosage forms may be used for oral, sublingual, or buccal administration. Solid unit dosage forms may be prepared by mixing the compounds with a pharmaceutically acceptable carrier and any other desired additives as described above. The mixture is typically mixed until a homogeneous mixture of the compounds and the carrier and any other desired additives is formed, i.e., until the compounds are dispersed evenly throughout the composition.

Tablets or pills can be coated or otherwise compounded to form a unit dosage form that has delayed and/or prolonged action, such as time release and sustained release unit dosage forms. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release.

The two components also may be separated by different layers that produce selective delivery of the agonist and the antagonist. The selective delivery is produced by differential solubilities of the layers in acid media or basic media. The coatings surrounding the compound to be delivered are dissolved in an ordered manner. The selective delivery allows for one compound to be released in a time and location dependent manner when compared to another compound present in the same formulation.

In another example, the tablet or pill can contain a polymer that controls the release of one component in the gastrointestinal tract and allows the second component to be release latter in the tract.

Biodegradable polymers for controlling the release of the compound, include, but are not limited to, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

The pharmaceutical composition or unit dosage forms of the present invention may be administered by a variety of routes such as oral, buccal, and sublingual. The pharmaceutical compositions or unit dosage forms of the present invention may be administered to an animal, preferably a human being.

The daily dosage of the compounds may vary according to a variety of factors such as underlying disease states, the individual's condition, weight, sex and age and the mode of administration. For oral administration, the pharmaceutical compositions can be provided in the form of scored or unscored solid unit dosage forms containing 0.01, 0.05, 0.1, 0.5, 1.0, 2.5, 5.0, 10.0, 15.0, 25.0, or 50.0 milligrams of the agonist and/or antagonist for the symptomatic adjustment of the dosage to the patient to be treated.

The dosage regimen utilizing the formulation of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the absorption, distribution, metabolism, and excretion of a drug.

The pharmaceutical composition or unit dosage form may be administered in a single daily dose, or the total daily dosage may be administered in divided doses of two, three or four times daily. In addition, co-administration or sequential administration of other active agents may be desirable. For combination treatment with more than one active agent, where the active agents are in separate dosage formulations, the active agents can be administered concurrently, or they each can be administered at separately staggered times. The dosage amount may be adjusted when combined with other active agents as described above to achieve desired effects. On the other hand, unit dosage forms of these various active agents may be independently optimized and combined to achieve a synergistic result wherein the pathology is reduced more than it would be if either active agent were used alone.

The formulation also may be administered as an additive to the feed by simply mixing the formulation with the feedstuff or by applying the formulation to the surface of animal feed. Alternatively, the formulation may be mixed with a carrier and the resulting composition may then either be mixed with the feed or fed directly to the animal. Suitable carriers include, but are not limited to, corn meal, citrus meal, fermentation residues, soya grits, dried grains, and the like. The formulation may be intimately mixed with the carrier by, e.g., grinding, stirring, milling, or tumbling.

Several methods may be used to produce the unit dosage forms and formulations of the present invention. In a specific embodiment, the amount of therapeutic agent present in the formulation is a therapeutic effective amount, i.e., that amount needed to produce a health benefit in the patient to which it is administered. Any method known to one of ordinary skill in the art may be used to prepare the formulations of the invention. In a specific embodiment, melt extrusion granulation or melt extrusion method are used to produce the dosage forms of the present invention.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that values are approximate, and are provided for description.

Patents, patent applications, publications, procedures, and the like are cited throughout this application, the disclosures of which are incorporated herein by reference in their entireties.

We claim:

1. An oral dosage form comprising a medicament and an insoluble component adhered to said medicament via a covalent linkage that is susceptible to cleavage by colonic bacteria and resistant to cleavage by intestinal saccharidases.

2. An oral dosage form of claim 1 wherein the insoluble component in a protein.

3. An oral dosage form of claim 2 wherein the protein is an immunoglobulin construct.

4. An oral dosage form of claim 3 wherein the immunoglobulin construct is a synthetic antibody.

5. An oral dosage form of claim 1 wherein the linkage is acid labile.

6. An oral dosage form of claim 5 wherein the linkage is susceptible to cleavage at a pH less than 4.

7. An oral dosage form of claim 1 wherein the linkage is susceptible to cleavage by an endogenous protease selected from the group consisting of pepsin, trypsin, and elastase.

8. An oral dosage form of claim 5 wherein the linkage is selected from the group consisting of an ester, an amide, a carbohydrate, a sulfonate, and a glucouronide.

9. An oral dosage form comprising a medicament and an insoluble component encapsulating the medicament, wherein the insoluble component is susceptible to dissolution and/or cleavage by colonic bacteria and resistant to cleavage by intestinal saccharidases.

10. An oral dosage form of claim 9 wherein the insoluble component in a protein.

11. An oral dosage form of claim 10 wherein the protein is an immunoglobulin construct.

12. An oral dosage form of claim 11 wherein the immunoglobulin construct is a synthetic antibody.

13. An oral dosage form of claim 9 wherein the insoluble component is acid labile.

14. An oral dosage form of claim 13 wherein the insoluble component is susceptible to dissolution and/or cleavage at a pH less than 4

15. An oral dosage form of claim 9 wherein the insoluble component is susceptible to dissolution and/or cleavage by an endogenous protease selected from the group consisting of pepsin, trypsin, and elastase.

16. An oral dosage form of claim 9 wherein the medicament is dispersed in a component selected from the group consisting of a pellet, bead, or microparticle, and said component is encapsulated by the insoluble component.
